(19) United States
(12) Patent Application Publication
(10) Pub. No.: US 2008/0097207 A1
(43) Pub. Date: Apr. 24, 2008

(54) Ultrasound therapy monitoring with diagnostic ultrasound

(75) Inventor: Anming He Cai, San Jose, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/520,274

(22) Filed: Sep. 12, 2006

(51) Int. Cl. A61B 8/00; A61H 1/00

(52) U.S. Cl. 600/442; 601/3

(57) ABSTRACT

The therapeutic ultrasound waveform is used as a source of stress or ARFI pushing pulse. When the therapeutic ultrasound waveform ceases, diagnostic ultrasound is used to measure the strain, such as measuring tissue displacement. The displacement over time after release of the stress indicates tissue characteristics. The tissue characteristics may be monitored to determine when sufficient therapeutic results are obtained.

### BACKGROUND

The present embodiments relate to monitoring acoustic therapy. In particular, ultrasound is used to monitor acoustic therapy.

High intensity focused ultrasound (HIFU) is used to treat cancers, tumors, lesions, or other undesired tissue structures. The ultrasound energy heats the tissue sufficiently to necrotize the undesired tissue. The ultrasound energy is focused to avoid harming healthy tissue. Ultrasound use may avoid invasive procedures, such as an operation or radio frequency ablation procedure.

Ultrasound imaging has been used to guide HIFU therapy. The imaging assists in focusing the therapy pulses on the undesired tissue. Attempts have also been made to monitor the thermal and biological changes of the tissue during these therapies. For example, ultrasound energy is used to measure thermal expansion coefficients (e.g., measure tissue expansion by speckle tracking), speed of sound in the tissue, or stiffness changes (e.g., strain imaging). However, these diagnostic based ultrasound tissue characterization may not have sufficient signal-to-noise resolution or may not be clinically viable.

For strain imaging, external pressure is applied to stress internal tissue. The response of the internal tissue to the application or release of the stress is measured with ultrasound energy. For example, correlation of B-mode data representing the tissue under different stress loads is used to determine tissue strain. For cardiac imaging, the strain rate may be determined using the heart motion as the source of stress.

Stress may be applied acoustically. Acoustic radiation force imaging (ARFI) exploits the stiffness difference between a lesion and surrounding tissues. For example, see U.S. Pat. No. 6,371,912, the disclosure of which is incorporated herein by reference. The radiation force of a strong pushing pulse induces micron level displacement of the target area. Due to diagnostic restrictions, the spatial peak time averaged intensity may not exceed 720 mW/cm². However, the signal-to-noise ratio (SNR) is limited by the pushing pulse intensity and length. Two-dimensional speckle tracking provides displacement over a millisecond period of tissue movement. The time resolution is limited by the imaging frame rate.

### BRIEF SUMMARY

By way of introduction, the preferred embodiments described below include methods, systems, improvements, and computer readable media for monitoring ultrasound therapy. The therapeutic ultrasound waveform is used as a source of stress or ARFI pushing pulse. When the therapeutic ultrasound waveform ceases, diagnostic ultrasound is used to measure the strain, such as measuring tissue displacement. The displacement over time after release of the stress indicates tissue characteristics. The intensity of the therapeutic pulse may result in better SNR and/or a greater displacement time. The tissue characteristics may be monitored to determine when sufficient therapeutic results are obtained.

In a first aspect, a method is provided for monitoring ultrasound therapy. Therapeutic ultrasound is transmitted to tissue. Ultrasound is used to measure displacement of the tissue based on cessation of the therapeutic ultrasound.

In a second aspect, a system is provided for monitoring ultrasound therapy. A transducer is operable to transmit diagnostic ultrasound. A trigger device is operable to trigger transmission of the diagnostic ultrasound in response to an end of a therapeutic ultrasound waveform. A processor is operable to determine strain as a function of echoes responsive to the triggered diagnostic ultrasound.

In a third aspect, a computer readable storage medium has stored therein data representing instructions executable by a programmed processor for synchronized ultrasound imaging and measurement. The storage medium includes instructions for measuring strain while tissue relaxes from stress applied by therapeutic ultrasound, and determining a value as a function of the strain.

In a fourth aspect, an improvement is provided in diagnostic ultrasound for determining tissue properties with acoustic force radiation. An acoustic pushing pulse applies stress to tissue, and ultrasound is used to measure the tissue response to the stress. The improvement is using therapeutic ultrasound as the pushing pulse.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for monitoring therapeutic ultrasound;

FIG. 2 is a graphical representation of one embodiment of an association of tissue strain with ultrasound;

FIG. 3 is a graphical representation of one embodiment of a sequence for therapy and imaging with ultrasound; and

FIG. 4 is a graphical representation of a system for monitoring therapy with ultrasound.

### DETAILED DESCRIPTION OF THE DRAWINGS AND PRESENTLY PREFERRED EMBODIMENTS

Acoustic force radiation imaging (ARFI) or other strain imaging is used to monitor the biological or temperature effects of high intensity focused ultrasound therapy (HIFU) or other acoustic therapy. HIFU waveforms provide radiation force or a pushing pulse to stress tissue. The high intensity focused ultrasound waveform may generate a much larger displacement in tissue than that generated by a diagnostic ultrasound system. Given the intensity for HIFU, ARFI is provided with a higher signal-to-noise ratio (SNR). When this pressure is released, the tissue displacement will decay over a few milliseconds. ARFI detects the tissue response or strain to the release of the acoustic pressure. The HIFU waveforms also generate biomechanical changes that can be detected by ARFI. By combining HIFU and ARFI, the ARFI may take advantage of the acoustic radiation force from the therapeutic ultrasound pressure.

In one embodiment, the therapeutic ultrasound used as a source of acoustic pressure has a spatial peak time averaged intensity, Iₛₚₜₐ, at or exceeding 1000 W/cm². The thermal effects of the therapy acoustic energy at such intensities may cause changes in volume due to thermal expansion, in the speed of sound (c), in tissue stiffness (E), and/or in the viscosity (η) of fluids in the tissue. The therapy acoustic energy may also induce mechanical effects, such as radiation pressure, streaming, and/or cavitations. The biological effects may include hyperthermiia at tissue temperature of about 41-45° C., protein denaturation at temperatures above 45° C., and tissue necrosis at temperatures above 50° C. Tissue stiffness may be effected even at temperatures below 45° C. At temperatures above 45° C., increases viscosity and/or stiffness may occur. At temperatures above 50° C., the tissue may have a high stiffness and/or high attenuation.

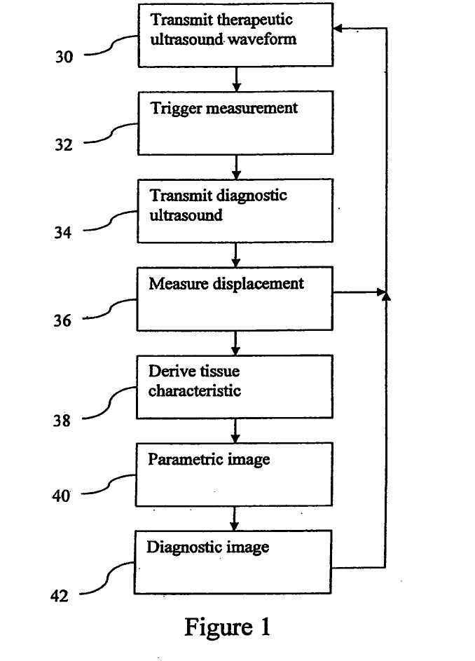

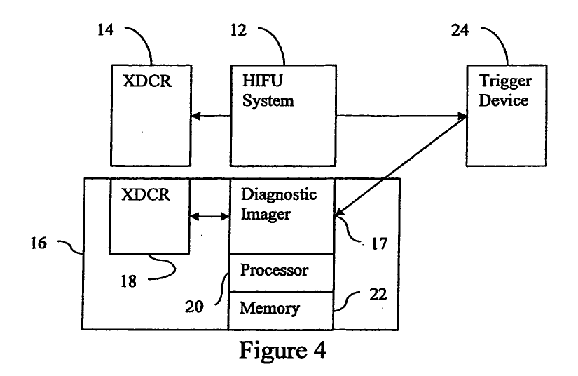

FIG. 1 shows a method for monitoring ultrasound therapy. The method is implemented by the system of FIG. 4 or a different system. The acts are performed in the order shown or a different order. Different, additional, or fewer acts may be performed. For example, acts 38, 40 and/or 42 are not performed.

Ultrasound imaging may be performed prior to therapy treatment. The lesion or other tissue for treatment is identified. The imaging and therapy systems are registered or share components, such as transducers or transducer housings. By aligning an imaging region with a therapy focus, the therapy system may be focused at the desired location. Any now know or later developed treatment preparation may be used.

In act 30, a therapeutic ultrasound pulse is transmitted. The pulse is focused using a phased array or mechanical focus and provides the high intensity acoustic energy to tissue at a treatment location. The therapeutic ultrasound pulse has a plurality of cycles at any desired frequency. In one embodiment, the therapeutic pulse lasts for a fraction of a second to seconds at an ultrasound frequency, such as 500 KHz-20 MHz. Any peak intensity may be provided, such as 100 or more watts per square centimeter, 500 or more watts per square centimeter, 1000-2000 watts per square centimeter, or about 1000 watts per square centimeter. Any now known or later developed therapeutic waveform with any intensity, frequency, and/or number of cycles may be used. The waveform is continuous or intermittent.

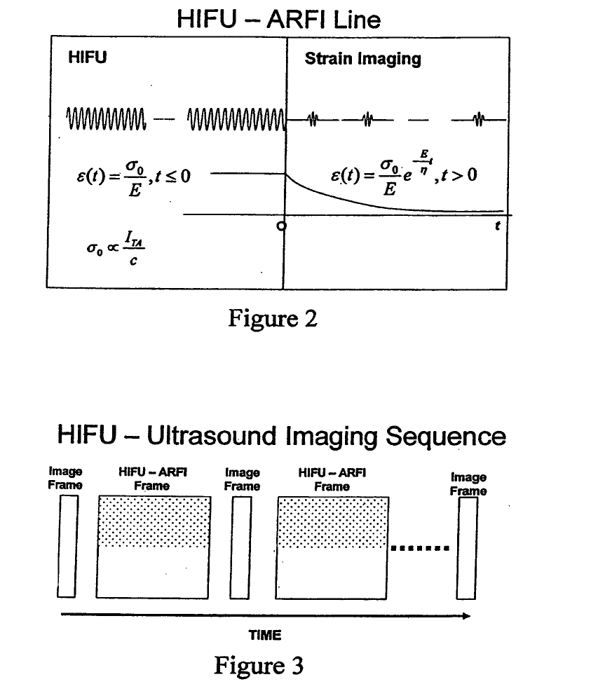

The therapeutic ultrasound pulse treats the tissue by generating heat at the desired tissue location. The intensity also generates stress on the tissue. The pulse pushes the tissue towards and away from the transducer with negative and positive acoustic pressures. For a sufficiently long therapeutic pulse, a substantially constant strain on the tissue is created. FIG. 2 shows a relatively long duration therapeutic pulse and the associated stress, σ, for HIFU. The strain, ε is a function of the tissue stiffness, E, the viscosity, η, and the stress from HIFU radiation force. The steady state stress during the therapeutic pulse is proportional to the ratio of average HIFU intensity, I, to the speed of sound in the tissue, c.

In act 32 of FIG. 1, measurement is triggered. The decay in stress and associated change in strain occurs upon the release of pressure by the therapeutic ultrasound. The decay may occur over a few milliseconds, but may decay over more or less time. The measurement is triggered to occur at least in part during the decay or change in stress and strain. Some of the measurements may occur during or interleaved with the therapeutic ultrasound and/or after the tissue reaches a substantially relaxed state.

The triggering is performed by sensing the cessation of the therapeutic pulse. Alternatively, the control that causes the cessation or a transmitter of the therapeutic ultrasound signals another controller or starts the measurement. A time or count down based trigger may be used. A counter may count a number of therapeutic pulse cycles. Other triggering may be used.

In act 34, one or more ultrasound pulses are transmitted for measurement. The pulses are transmitted from a same or different transducer as the therapeutic pulses. The pulses have any desired amplitude and duration, such as relatively short duration pulses having a therapeutic intensity. In one embodiment, the measurement pulses are diagnostic pulses, such as having an intensity and duration below the regulated levels for diagnostic ultrasound. For example, pulses with 1-5 cycle durations are used with an intensity of less than 720 mW/cm². Pulses with other intensities may be used, such as pulses with less than 1000 mW/cm².

The ultrasound transmission is focused at the same tissue as the therapeutic ultrasound. The transmission may cover one or more scan lines. For example, a wide beam width transmit pulse is used for receiving along two or more receive scan lines with a plane or volume distribution. Alternatively, a single receive beam is formed in response to a transmit. A region may be sequentially scanned where more than one transmit event is possible during the decay time. One or more measurements are performed for each receive scan line.

Two or more, such as 2-10, pulses are transmitted to a same location for each measurement or for combining measurements. Alternatively, a single pulse may be transmitted for each measurement. Where the therapeutic intensity and time since cessation are known, a single pulse may be used and compared to pre-HIFU measurement to determine a rate or decay characteristic of the strain.

In act 36, the displacement of the tissue is measured. The echoes from the transmitted ultrasound are used for measuring the displacement of the tissue. After cessation of the therapeutic ultrasound, the tissue moves to a relaxed position. Echoes from the multiple relatively low diagnostic imaging pulses are fired after the long therapeutic waveform. The echoes are used to generate one or more ARFI images to track the displacement-time curve or strain associated with the release of the stress.

The echoes are detected using B-mode or Doppler detection. Using B-mode data, the data from multiple pulses is correlated. The correlation is one, two or three-dimensional. For example, correlation along a scan line away and toward the transducer is used. Any now known or later developed correlation may be used, such as cross-correlation, pattern matching, or minimum sum of absolute differences. Tissue structure and/or speckle are correlated. Using Doppler detection, a clutter filter passes information associated with moving tissue. The velocity of the tissue is derived from multiple echoes. The velocity is used to determine the displacement towards or away from the transducer. Alternatively, the relative or difference between velocities at different locations may indicate strain or displacement.

The amount displacement represents tissue characteristics given a therapeutic intensity. The initial displacement may be proportional to the intensity of the therapeutic waveform. The time associated with a particular displacement allows estimation of the decay curve shown in FIG. 2. By measuring the displacement as a function of time, the decay of strain from cessation of the therapeutic ultrasound may be measured. Displacement alone or any characteristic of the decay may be measured.

In act 38, one or more tissue characteristics may be derived from the measured displacement and/or decay curve determined from the measured displacement. The derivation is a calculation or from a look-up table. For example, displacement at a particular time after cessation of the therapeutic ultrasound or other characteristic of the decay curve indicates a particular tissue characteristic. Experimentation may indicate the relationship of one or more characteristics to measured values or combinations of values. Tissue stiffness, viscosity, temperature, thermal expansion, and/or speed of sound may be derived from the displacement. For example, the tissue stiffness is calculated from the displacement and the intensity of the therapeutic ultrasound. The decay time is proportional to the viscosity of the fluid in the tissue. This viscosity is correlated with the protein denaturation process and the inverse of the stiffness. Viscosity may be related to tissue necrosis.

In act 40, a parametric image is generated. Image values are assigned based on the displacement, decay characteristic (e.g., decay time), and/or derived information. The HIFU-ARFI lines or data are combined to form monitoring images for display. The images can be the displacement themselves or/and the derived parameters that represent biological or/and temperature changes. For example, the brightness, color, or other image information is modulated as a function of the displacement or derived information. More than one image characteristic may be modulated, such as the brightness being modulated by strain or stiffness and the color being modulated by temperature. Interpolation may be used. The parametric image may be for a region of interest, such as a region of the therapy tissue, or for a larger region, such as a diagnostic imaging region.

In act 42, a diagnostic image is generated. B-mode, Doppler, B-mode and Doppler, and/or other diagnostic ultrasound images may be generated. The diagnostic image is generated for a one, two or three-dimensional region. The region covers the tissue to be treated.

The diagnostic image is generated after measurement. In one embodiment, a single therapy event is provided. In other embodiments, the therapy is repeated multiple times during a session. Acts 30, 32, 34, and 36 are repeated. Other acts may be repeated, such as also repeating acts 38, 40 and/or 42. The repetition occurs until the desired therapeutic effect occurs. The raw displacement or a parameter derived from the displacement is compared to a threshold or other information to determine whether the desired effect has occurred. Alternatively, a user indicates an appropriate time to cease the process.

The whole treatment process starts with a diagnostic imaging guide for placing the therapeutic ultrasound, followed by the HIFU treatment and the continuous monitoring the biological and/or thermal changes during the treatment. Once the targeted biological and/or thermal effects are identified by the monitoring images, the treatment is stopped, and the result is further confirmed by a complete set of diagnostic images.

FIG. 3 shows the repeating sequence. The HIFU-ARFI frame includes acts 30, 32, 34, and 36. The shading represents the therapy portion and the non-shaded area represents the measurement portion. The image frame includes act 42. During the repetition, the diagnostic images may be used to track movement caused by patient's breathing and other motions. The tracking allows alignment of the therapy focus with the tissue to be treated.

The parametric image may be combined with the diagnostic image. For example, a strain image is overlaid on a B-mode and/or Doppler image. The HIFU-ARFI imaging frames generated from the HIFU-ARFI data are further combined with typical diagnostic imaging frames, which include B-mode, color-Doppler, contrast-pulse sequence perfusion imaging or other imaging modes providing diagnostic imaging and motion correction. Other corrections for large-scale speed of sound change, acoustic attenuation, and thermal expansion can also be made through the information obtained from the diagnostic and/or the HIFU-ARFI image frames.

FIG. 4 shows a system, the diagnostic imaging system 16, for monitoring ultrasound therapy. The system 16 implements the method of FIG. 1 or a different method. A therapy system 12 is separately provided with the diagnostic imaging system 16. While shown separately, the therapy and diagnostic imaging systems 12, 16 may be combined in a same system with or without shared components.

The therapy system 12 includes the transducer 14. In one embodiment, the therapy system 12 is a high intensity focused ultrasound system. A beamformer generates waveforms and relatively delays the waveforms. The transducer 12 is a array for generating acoustic energy from the waveforms. The relative delays focus the acoustic energy. A given transmit event corresponds to transmission of acoustic energy by different elements at a substantially same time given the delays. The transmit event provides a pulse of ultrasound energy for treating the tissue. Alternatively, a mechanical focus is provided. Any now known or later developed therapy system 12 and transducer 14 may be used.

The diagnostic imaging system 16 includes a diagnostic imager 17, a transducer 18, a processor 20, and a memory 22. Additional, different or fewer components may be provided. For example, the processor 20 and/or memory 22 are part of the therapy system 12 and/or are separate from the imaging system 16.

The transducer 18 is an array of elements. One, two or multi-dimensional arrays may be used. Piezoelectric or cMUTs may be used. The transducer 18 is sized and shaped for transmission and reception of diagnostic ultrasound, such as acoustic energy with relatively low intensity.

In one embodiment, the transducer 18 is separate from the transducer 14. Imaging is used to determine the therapy location. Alternatively, both transducers 14, 18 include spatial registration systems, such as magnetic position sensors. Alternatively, the transducers 14, 18 are connected together, such as being positioned in a same housing. In other embodiments, the transducers 14, 18 are the same device. One or more elements are used for both therapy and diagnostic transmissions.

The diagnostic imager 17 includes a beamformer, a detector (e.g., B-mode and/or Doppler), a scan converter, and a display. Additional, different or fewer components may be provided, such as including filters. The diagnostic imager 17 generates transmit waveforms for scanning with the transducer 18. The transducer 18 converts echoes into electrical signals for beamformation by the imager 17. The beamformed data is detected and used for monitoring the therapy or imaging. In one embodiment, the imager 17 includes a B-mode detector operable to generate B-mode or intensity data in response to the echoes. In another embodiment, the imager 17 includes a Doppler detector operable to estimate velocities or other tissue movement in response to the echoes. The imager 17 includes any now know or later developed components for implementing any strain, elasticity, or ARFI imaging.

The processor 20 is a control processor, general processor, digital signal processor, application specific integrated circuit, field programmable gate array, graphics processor, Doppler processor, digital circuit, analog circuit, combinations thereof, or any other now known or later developed device for determining strain or correlating. The processor 20 is part of the imaging system 20, but may be part of the therapy system 12 or separate from both. The processor 20 controls operation of the imager 17, the therapy system 12 or both.

Alternatively or additionally, the processor 20 determines strain or displacement as a function of echoes. The imager 17 transmits a sequence of pulses, such as diagnostic pulses. Data detected from responsive echoes are used to determine displacement. Strain may be determined as a function of the displacement of tissue. In one embodiment, the processor 20 correlates B-mode data from different transmit events. By searching for a best or sufficient fit in one, two, or three dimensions, an amount of displacement between the different transmit events is determined. In another embodiment, Doppler estimates are generated from echoes generated from different transmit events. For example, velocity is estimated. The velocity and time may be used to determine a displacement. Alternatively, strain is directly estimated based on the velocity.

The transmit events for determining displacement are timed relative to the therapeutic waveforms. The timing allows use of the therapeutic waveforms as a source of tissue stress or pressure for measuring displacement. The trigger device 24 provides the timing information to the imaging system 16.

The trigger device 24 is a processor, switch, counter, register, timer, delay, digital circuit, analog circuit, combinations thereof, or other device operable to indicate timing information. The trigger device 24 is part of the therapy system 12, the diagnostic imaging system 16, both, or is separate from both. In one embodiment, the trigger device 24 is a controller of the therapy system 12, the imaging system 16, or both. A signal to cease generation of the therapy waveform is also used to trigger diagnostic transmissions. In other embodiments, the trigger device 24 is a transmitter of the therapy system 12, and outputs a signal at or before cessation. The trigger device 24 may sense acoustic energy or transmit waveforms of the therapy system 12 and output a trigger signal based on the sensed information. The trigger device 24 may count down to or time output of a trigger based on previous input information from the therapy system 12.

The trigger device 24 outputs a signal timed to the cessation of the therapeutic waveform or an indication of when the waveform will cease. The output is before, in correspondence with, or after cessation. The information from the trigger device 24 triggers transmission of the diagnostic ultrasound in response to an end of a therapeutic ultrasound waveform. A sequence of pulses of the diagnostic ultrasound is triggered. The triggered sequence to a same location or same scan line occurs within five or fewer milliseconds. The trigger signal is provided early enough that at least part of the sequence is transmitted during the decay time and before the tissue reaches a substantially relaxed position.

The memory 22 is a computer readable storage medium, such as a cache, buffer, register, RAM, removable media, hard drive, optical storage device, or other computer readable storage media. Computer readable storage media include various types of volatile and nonvolatile storage media. The memory 22 is part of the imaging system 16, the therapy system 12, or separate from either system 12, 16. The memory 22 is accessible by the processor 20.

In one embodiment, the memory 22 stores data for use by the processor 20, such as storing detected and/or image data for determining displacement. Additionally or alternatively, the memory 22 stores data representing instructions executable by the programmed processor 20 for monitory therapy. The instructions for implementing the processes, methods and/or techniques discussed herein are provided on computer-readable storage media or memories. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

In one embodiment, the instructions are for measuring strain while tissue relaxes from stress applied by therapeutic ultrasound. The processor causes transmission of a plurality of relatively low intensity pulses in response to cessation of relatively high intensity therapeutic ultrasound pulse. The processor determines displacement of the tissue from data responsive to the transmission. Strain may be determined from the displacement. Imaging or tissue characteristic values may be determined from the strain or displacement. For example, a tissue stiffness, viscosity, temperature, thermal expansion, or speed of sound is determined.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for monitoring ultrasound therapy, the method comprising:
transmitting a therapeutic ultrasound to tissue at a location; and
measuring, with ultrasound, displacement of the tissue in response to cessation of the therapeutic ultrasound.

2. The method of claim 1 wherein transmitting comprises transmitting high intensity focused ultrasound operable to treat the tissue by heat generation, the location being within a patient.

3. The method of claim 1 wherein measuring comprises tracking the displacement with ultrasound B-mode information.

4. The method of claim 1 wherein measuring comprises correlating speckle as a function of time.

5. The method of claim 1 wherein transmitting comprises generating stress on the tissue with the therapeutic ultrasound as a pushing pulse, and wherein measuring comprises generating an acoustic radiation force image representing strain associated with the stress.

6. The method of claim 1 wherein measuring displacement comprises measuring the displacement as a function of time.

7. The method of claim 6 wherein measuring displacement as a function of time comprises determining a change of strain from cessation of the therapeutic ultrasound.

8. The method of claim 1 further comprising:
deriving tissue stiffness, viscosity, temperature, thermal expansion, speed of sound, or combinations thereof as a function of the displacement.

9. The method of claim 1 further comprising:
diagnostically imaging the tissue after measuring; and
repeating the transmitting, measuring, and imaging for the tissue as a function of the displacement.

10. The method of claim 1 further comprising:
generating a parametric image as a function of the displacement; and
displaying (a) the parametric image and (2) a B-mode image, a Doppler image, or both.

11. A system for monitoring ultrasound therapy, the system comprising:
a transducer operable to transmit diagnostic ultrasound;
a trigger device operable to trigger transmission of the diagnostic ultrasound in response to an end of a therapeutic ultrasound waveform; and
a processor operable to determine strain as a function of echoes responsive to the triggered diagnostic ultrasound.

12. The system of claim 11 wherein the trigger device comprises a controller operable to control the application of the therapeutic ultrasound waveform and transmission of the diagnostic ultrasound.

13. The system of claim 11 wherein the therapeutic ultrasound waveform has an intensity exceeding 100 W/cm² and the diagnostic ultrasound has an intensity less than 1000 mW/cm².

14. The system of claim 11 wherein the trigger device is operable to trigger transmission of a sequence of pulses of the diagnostic ultrasound to a location within five or fewer milliseconds, and wherein the processor is operable to determine the strain as a function of echoes responsive to the sequence of pulses.

15. The system of claim 11 wherein the processor is operable to determine the strain as a function of displacement of tissue represented by the echoes.

16. The system of claim 15 further comprising:
a B-mode detector operable to generate B-mode data with the echoes;
wherein the processor is operable to determine the displacement as a function of correlation of the B-mode data.

17. In a computer readable storage medium having stored therein data representing instructions executable by a programmed processor for monitoring ultrasound therapy, the storage medium comprising instructions for:
measuring strain while tissue relaxes from stress applied by therapeutic ultrasound and beginning the measuring before the tissue reaches a relaxed state; and
determining a value as a function of the strain.

18. The instructions of claim 17 wherein measuring strain comprises:
transmitting a plurality of relatively low intensity pulses in response to cessation of relatively high intensity therapeutic ultrasound pulse; and
determining displacement of the tissue.

19. The instructions of claim 17 wherein determining the value comprises determining an imaging value.

20. The instructions of claim 17 wherein determining the value comprises determining at least a tissue stiffness, viscosity, temperature, thermal expansion, or speed of sound.

21. In diagnostic ultrasound for determining tissue properties with acoustic force radiation wherein an acoustic pushing pulse applies stress to tissue and ultrasound is used to measure the tissue response to the stress, an improvement comprising:
using therapeutic ultrasound as the pushing pulse.
